(19) United States
(12) Patent Application Publication
(10) Pub. No.: US 2010/0028969 A1
(43) Pub. Date: Feb. 4, 2010

(54) CELL LYSIS OR ELECTROPORATION DEVICE COMPRISING AT LEAST ONE PYROELECTRIC MATERIAL

(75) Inventors: Manfred Mueller, Eindhoven (NL); Mark Thomas Johnson, Veldhoven (NL); Murray Fulton Gillies, Eindhoven (NL); Marc Wilhelmus Gijsbert Ponjee, Tilburg (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/518,898

(22) PCT Filed: Dec. 14, 2007
(86) PCT No.: PCT/IB2007/055116

§ 371 Date: Jun. 12, 2009

(30) Foreign Application Priority Data
Dec. 18, 2006 (EP) 06126392.7

(51) Int. Cl. C12N 13/00; C12M 1/33; C12M 1/34

(52) U.S. Cl. 435/173.7; 435/306.1; 435/287.2; 435/287.1

(57) ABSTRACT

The present invention relates to a cell lysis or electroporation device comprising at least one pyroelectric material.

The present invention is directed to the field of devices for the lysis or electroporation of biological cells.

The process of rupturing a cell membrane is called lysing (or lysis). Lysing can be performed by various chemical, mechanical or electrical methods, all of which can result in the rupturing of the cell membrane.

The membrane of cells is primarily composed of two layers of phospholipid molecules. These bipartite molecules have hydrophilic and hydrophobic ends. The hydrophilic ends are directed to the inside and the outside surfaces of the membrane and the hydrophobic towards the inner surface. The ease with which this membrane can be ruptured varies widely between different cell types. For some cells a simple salt solution can be sufficient to cause a breakdown of the membrane. With other cells, however, a mechanical lysing step is necessary where the cells are milled with, for example, silica and/or ceramic beads to release the contents.

In the most hardy of cells such as, Gram-positive, yeast cells or fungus cells mechanical lysing is usually not sufficient and enzymatical lysing is necessary. In this case, specially prepared enzymes break down the cell membrane. These enzymes can lyse nearly all types of cells but are intrinsically expensive to produce and are unstable if not used quickly after preparation.

Recently electrical lysing has been attracting attention. This uses pulsed electrical fields in order to rupture the cell membrane. If, however, the electric field is not sufficient to rupture the membrane then the membrane may become more porous and allow other molecules to enter which would otherwise be blocked (i.e. medicines or other DNA (in the case of transinfection)). Such a reversible increase in porosity due to an electrical field is known as electroporation and the cell must be viable after the process.

It is therefore an object of the present invention to provide a device, which is able to at least partly overcome the above-mentioned drawbacks and to provide a possibility of reliable cell lysis or electroporation with less side effects.

This objective is solved by a device according to claim 1 of the present invention. Accordingly, a cell lysis or electroporation device is provided comprising a pyroelectric material.

Surprisingly it has been found that by such a material, an electrical field can be generated which lyses cells for a wide range of applications within the present invention in a reliable manner with only few side effects or with no side effects at all.

By using such a device at least one or more of the following advantages can be achieved for a wide range of applications within the present invention.
The current present during the lysis can be greatly reduced, thus greatly limiting side effects thus as destruction or degradation of the material inside the cell.
The invention allows electrical lysing to be carried out at lower voltages than is currently possible with the state of the art devices.
The present invention furthermore allows electrical lysing to be carried out on cells or bio particles which are currently too robust to be lysed/porated via electrical fields.
The device can be designed in such a way that no further parts or materials (such as enzymes) have to be added before or after each lysis, thereby allowing a high-throughput strategy.
Since most pyroelectric materials are comparatively inexpensive and can be easily integrated into large area electronic (LAE) platforms and no outside components are needed, it is possible to construct very highly integrated inexpensive devices.

The present invention provides in a first aspect a device for the lysis or electroporation of biological cells. As will be appreciated by those in the art, these cells may be, but not limited to intact cells, cellular fragments such as membrane fragments, cellular organelles, bacteria, viruses, protozoa, and the like.

The term “pyroelectric material” in the sense of the present invention especially means and/or includes any material that exhibits a change of the spontaneous electrical polarization upon a change in temperature.

According to a preferred embodiment of the present invention, the pyroelectric material is a pyroelectric crystal material.

According to a preferred embodiment of the present invention, the pyroelectric material is provided at least partially in form of one or more deposited layer (s).

According to a preferred embodiment of the present invention the device furthermore comprises a temperature change means, which is preferably provided in the vicinity of at least one pyroelectric material.

According to a preferred embodiment of the present invention at least one pyroelectric material has an absolute value of the change of charge greater than ≧0.05 mC per ° C. per m², preferably ≧0.1 mC per ° C. per m² to ≦0.6 per ° C. per m².

According to a preferred embodiment of the present invention the device furthermore comprises at least one conductive material, which is shaped in a way to increase the electrical field and/or to allow charge transport to and from the pyroelectric material. This has been shown to be advantageous for a wide range of applications within the present invention as will e.g. be apparent from some of the embodiments to be discussed.

According to a preferred embodiment of the present invention the temperature change means comprises a cooling element, preferably a Peltier element. It has been shown for a wide range of applications within the present invention, that this may lead to a further “freeze”-effect within the cells, which may facilitate the lysis.

According to a preferred embodiment of the present invention the temperature change means comprises a structured resistive heating element for selective heating of parts of the at least one pyroelectric material. It will be apparent for the skilled person in the art, that this heating element may comprise a Peltier element as well.

According to a preferred embodiment of the present invention the temperature change means comprises a radiative means (e. g. a laser or microwaves) for selective heating of parts of the at least one pyroelectric material.

According to a preferred embodiment of the present invention the at least one pyroelectric material is selected out of the groups:

a) titanates or zirconate titanates, preferably of the structure M^{1I}[ZrₓTi₁₋ₓ]O₃ with 0≦x≦1 and M^{1I} selected out of the group comprising Pb, Be, Mg, Ca, Sr, Ba or mixtures thereof,

b) tantalates, preferably of the structure M^{I}TaO₃ with M¹ selected out of the group comprising Li, Na, K, Rb, Cs or mixtures thereof,

c) niobates, preferably of the structure M^{I}NbO₃ with M¹ selected out of the group comprising Li, Na, K, Rb, Cs or mixtures thereof, or

d) mixtures thereof.

The present invention furthermore relates to a method of lysing or electroporating of cells comprising the step of applying an electric field generated by a temperature change of at least one pyroelectric material.

The present invention furthermore relates to the use of a pyroelectric material for the lysis or electroporation of cells.

A device according to the present invention may be of use in a broad variety of systems and/or applications, amongst them one or more of the following:
biosensors used for molecular diagnostics
biosensors where cellular components have to be extracted
biosensors where the porosity of the cell membrane has to be altered
rapid and sensitive detection of proteins and nucleic acids in complex biological mixtures such as e.g. blood or saliva
high throughput screening devices for chemistry, pharmaceuticals or molecular biology
testing devices e.g. for DNA or proteins e.g. in criminology, for on-site testing (in a hospital), for diagnostics in centralized laboratories or in scientific research
tools for DNA or protein diagnostics for cardiology, infectious disease and oncology, food, and environmental diagnostics
tools for combinatorial chemistry
analysis devices
drug delivery devices for medical applications

The aforementioned components, as well as the claimed components and the components to be used in accordance with the invention in the described embodiments, are not subject to any special exceptions with respect to their size, shape, material selection and technical concept such that the selection criteria known in the pertinent field can be applied without limitations.

### BRIEF DESCRIPTION OF THE DRAWINGS

Additional details, features, characteristics and advantages of the object of the invention are disclosed in the subclaims, the figures and the following description of the respective figures and examples, which—in an exemplary fashion—show several preferred embodiments of a device according to the invention.

FIG. 1 shows a very schematic cross-sectional partial view showing a device according to a first embodiment of the present invention with two pyroelectric materials;

FIG. 2 shows a very schematic cross-sectional partial top view showing a device according to a second embodiment of the present invention with two pyroelectric materials;

FIG. 3 shows a very schematic top view of a slightly altered device of FIG. 2 with heating means;

FIG. 4 shows a very schematic cross-sectional partial view showing a device according to a third embodiment of the present invention with two pyroelectric materials and two conducting materials in form of a tip;

FIG. 5 shows the device of FIG. 4 with field lines;

FIG. 6 shows a very schematic cross-sectional partial view showing a device according to a fourth embodiment of the present invention;

FIG. 7 shows a very schematic cross-sectional partial view showing a device according to a fifth embodiment of the present invention with two pyroelectric materials and spacers between the pyroelectric materials;

FIG. 8 shows a very schematic partial top view showing a device according to a sixth embodiment of the present invention with a laser heating means, viewed along the line IV-IV in FIG. 9;

FIG. 9 shows a very schematic partial cross-sectional view of the device in FIG. 8 along line II-II in FIG. 8;

FIG. 10 shows a very schematic partial top view showing a device according to a seventh embodiment of the present invention, viewed along the line V-V in FIG. 11;

FIG. 11 shows a very schematic partial cross-sectional view of the device of FIG. 10 along line III-III in FIG. 10; and

FIG. 12 shows an active matrix heater array for use with a device according to the present invention.

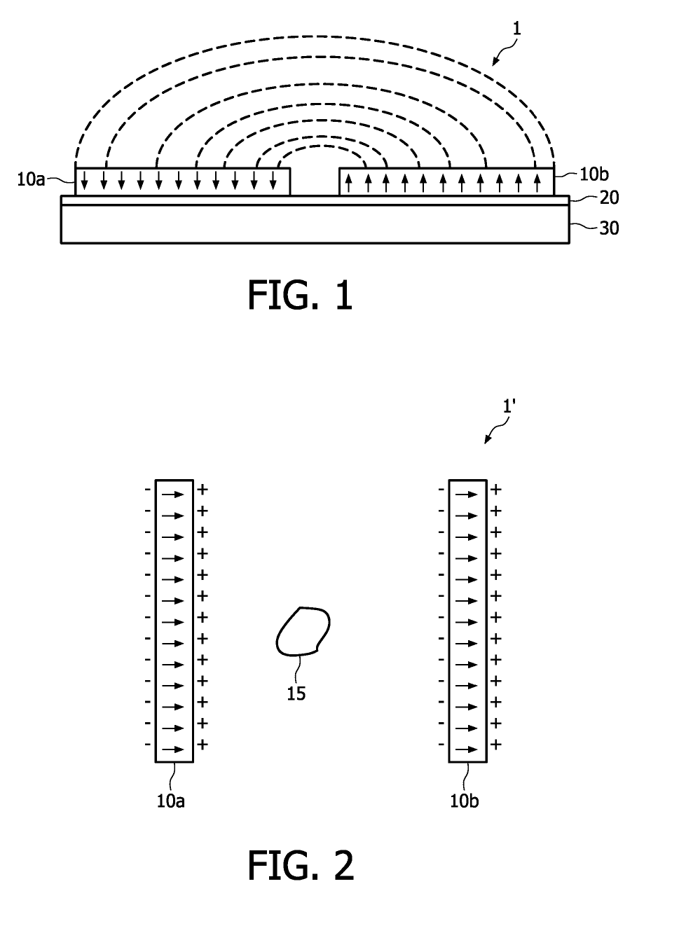

FIG. 1 shows a very schematic cross-sectional partial view showing a device 1 according to a first embodiment of the present invention with two pyroelectric materials 10a, 10b which are located on top of a conductive layer 20, which is itself placed on top of a heating layer 30. The arrows indicate the direction and orientation of the crystallographic axis (z-axis) of the pyroelectric material(s).

If the heating layer 30 changes the temperature of the pyroelectric materials 10a, 10b, opposite charges gather at the +z and −z faces of the crystals, generating an electrical field as indicated by the dashed lines. The conductive layer 20 is in this embodiment used to short-circuit one face of the crystals.

The charge per area generated is proportional to the temperature change and the pyroelectric properties of the pyroelectric crystal. However, over time charge will leak away, so the electrical field achievable is determined by the rate of temperature change as well as the magnitude of the temperature change and the properties of the pyroelectric crystal. By sending current pulses through the heaters, high E-field pulses can be generated by the pyroelectric material. To control the generated electrical field, in particular the temperature change may be controlled. Parameters that can be used to control this are the signal pulse height, signal pulse width, frequency of the pulses.

It should be noted that (not shown in the Figs) instead of using two or more pyroelectric materials it is possible to use one pyroelectric material with multiple ferroelectric domains, which is actually one further embodiment of the present invention.

However, it should be stressed that for a more simple layout (not shown in the Figs) it is also possible to replace one pyroelectric material in the figure with a simple (metallic) electrode, so that it is actually possible to carry out the invention with only one single pyroelectric material, which is actually a further preferred embodiment of the present invention. It is remarked that such a replacement in FIG. 1 would reduce the electric field by ½.

The heating layer may be part of a temperature controlled region on the device, which may comprise a temperature sensor. Optionally, the heating layer consists of an array of heated (or temperature controlled) segments, which may be controlled individually and in parallel.

According to a further embodiment of the present invention and especially for applications where currents are to be avoided, which may lower the charge (and therefore the field) as well as being the cause of (undesired) electrolysis, according, the pyroelectric materials are coated with a resistive layer.

However, also uncoated pyroelectric materials have proven themselves in practice for a large range of applications within the present invention, especially if very short pulses are desired.

FIG. 2 shows a very schematic partial top view showing a device 1′ according to a second embodiment of the present invention with two pyroelectric materials 10a, 10b.

In all drawings, for the sake of clearness and readability, the (essentially) same components and/or materials are referred to with the same numbers.

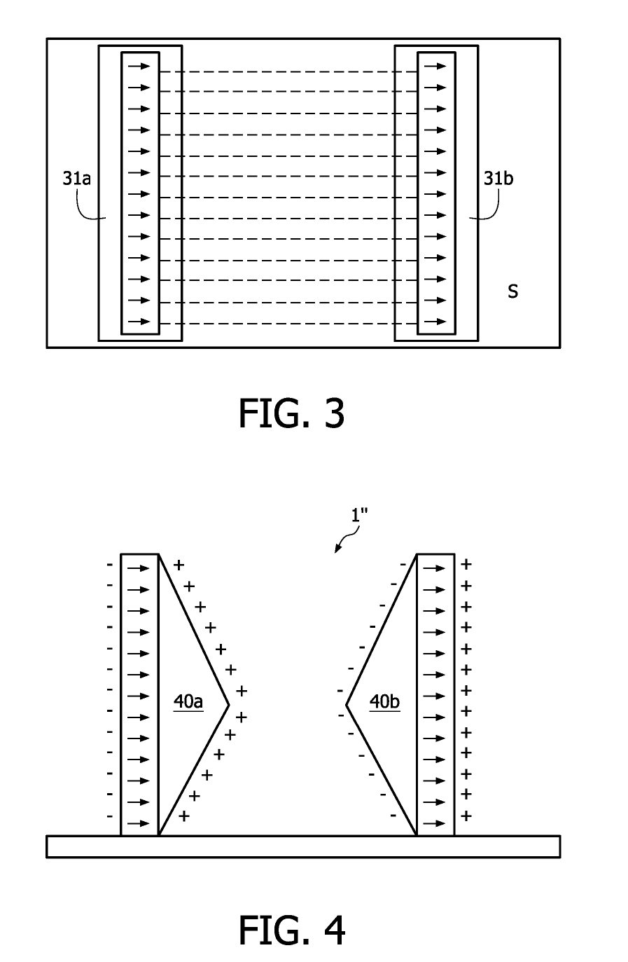

In FIG. 2—although not drawn explicitly in this Fig. but in FIG. 3—the field will follow “from left to right”, causing lysis or electroporation e.g. of a cell 15 which is located between the two pyroelectric materials. The field lines, however, can be seen in FIG. 3.

FIG. 3 shows a device similar to the device in FIG. 2 where the pyroelectric material forms thin layers with the z-axis parallel to the substrate S, which allows very efficient fabrication of the device in a single patterning step. Furthermore, several heating means 31a, 31b have been introduced.

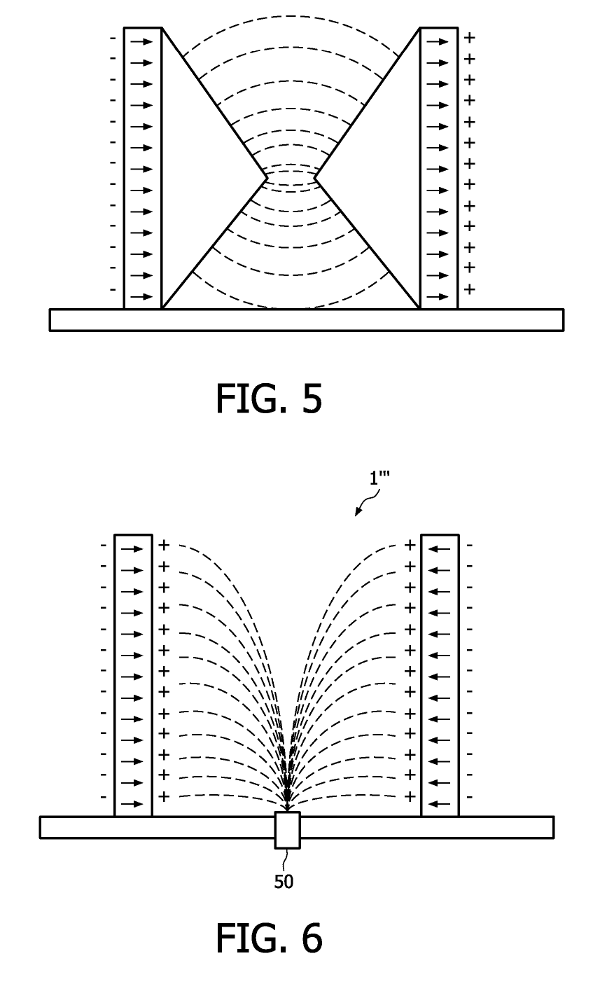

FIG. 4 shows a very schematic cross-sectional partial view showing a device 1″ according to a third embodiment of the present invention with two pyroelectric materials and two conducting materials 40a, 40b in form of a tip. As will be apparent from FIGS. 4 and 5, in this embodiment, the strongest field will be present between the two tips of the conducting materials 40a, 40b. Such a layout has proven itself in practice for a large range of applications within the present invention.

FIG. 5 shows the device of FIG. 4 with field lines introduced.

FIG. 6 shows a very schematic cross-sectional partial view showing a device 1″′ according to a fourth embodiment of the present invention. In this device, the orientation of one of the two pyroelectric materials is reversed and one central electrode 50 is introduced. This will cause the field lines to lead from the pyroelectric materials to the electrode rather than leading from one pyroelectric material to the other. By specially shaping the electrode 50 e.g. in the form of a tip, high field strengths can be achieved.

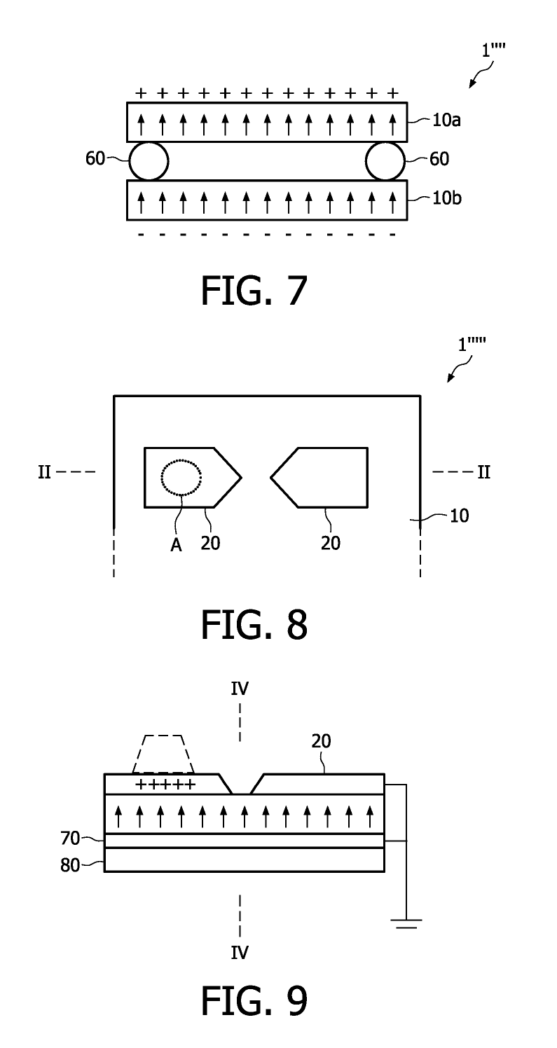

FIG. 7 shows a very schematic cross-sectional partial view showing a device 1″″ according to a fifth embodiment of the present invention with two pyroelectric materials and spacers between the pyroelectric materials. Such a simple sandwich structure has shown to be beneficial especially in throughput (and high-throughput) setups of the device according to the present invention.

Usually a liquid or fluid containing the cells to be lysed or electroporated is transported in the cavity between the two pyroelectric materials; the size of the spacers 60 will preferably be matched to the sort of cells which are to be lysed or electroporated and the time the liquid or fluid will stay in the cavity between the two pyroelectric materials as well as the velocity of the flow.

Below or above the two pyroelectric materials (not shown in the Figs.) there may be—according to the actual application—heating layers and/or first one or more conductive layers and then a heating layer. Of course, it may also be possible to replace one pyroelectric material by an electrode as described above.

FIG. 8 shows a very schematic partial top view showing a device 1″″′ according to a sixth embodiment of the present invention with a laser heating means (viewed along line IV-IV in FIG. 9). FIG. 9 shows a very schematic partial cross-sectional view of the device of FIG. 8 along line II-II in FIG. 8.

In this device, as will be apparent from FIG. 9, on top of a layer of pyroelectric material 10 several layers of conductive material 20 (in “tip”-form, as can be seen in FIG. 8) are provided. Below the layer of pyroelectric material 10, a transparent conductive material 70, preferably fabricated from a material such as ITO, and a transparent substrate 80 are provided. A microfluidic channel may be so constructed to specifically guide the cells over the tip. Alternatively, an array of tips with staggered gaps may be used to increase the chance of a cell passing over a tip.

The embodiment of FIGS. 8 and 9 is used for many applications, where the cells should not be exposed to large increases in temperature. I.e., when electroporation is performed it is for some applications essential that the cell stays viable and therefore the proteins attached to the cell membrane should not denature. To avoid heating the sample during lysis or electroporation pulses of strong E-field can be produced by rapidly heating the pyroelectric layer 10. This is done via irradiating the layer with a laser beam in the area indicated with an “A” in FIG. 8. Since the laser beam (indicated as the trapezoid in FIG. 9) is pulsed and can be strongly focused into the pyroelectric layer there is little heating of the sample fluid. It should be noted that for some applications the laser beam may be directed to the pyroelectric layer “from below”, i.e. through the substrate 80 and the transparent conductive layer 70.

The embodiment of FIGS. 8 and 9 allows for a wide range of applications within the present invention because the temperature of the pyroelectric material, and with that the generated E-field, may be well controlled using parameters such as the pulse duration, pulse rate, or laser intensity.

To facilitate heating by a laser beam for a wide range of applications the pyroelectric layer is—according to a further embodiment of the present invention and not shown in the Figs.—tuned to absorb the wavelength of the laser. While some pyroelectric crystals like lithium tantalate are naturally transparent, it is possible to dope the material with light absorbing ions without significantly changing the pyroelectric behaviour. It is therefore easy to create any desired degree of opacity (in fact it might be desirable to create a concentration gradient, to create a very homogeneous heating across the crystal width or to heat only very locally).

Alternatively according to a further embodiment of the present invention and not shown in the Figs a light-absorbing layer that transforms the laser light into heat is put in good thermal contact with the pyroelectric material. Optionally, a heatsink layer (as e.g. present in re-writable DVD disks based on phase change materials) is present in good thermal contact with the heating layer and/or pyroelectric layer, to optimise the generated electric field pulse shape or to enable a higher E-field pulse frequency.

Using optics originally developed for DVD applications it is possible to strongly focus (infrared or visible) laser light and to rapidly heat up areas of the pyroelectric material for a wide range of applications within the present invention. This is a further embodiment of the present invention.

This strong localization has for a wide range of applications within the present invention the additional advantage that the sample itself may be placed well outside of the heating area and would therefore remain unaffected by the increase in temperature or the high light intensity. It has been shown for a wide range of applications within the present invention that a setup according to FIGS. 8 and 9 also facilitates cooling.

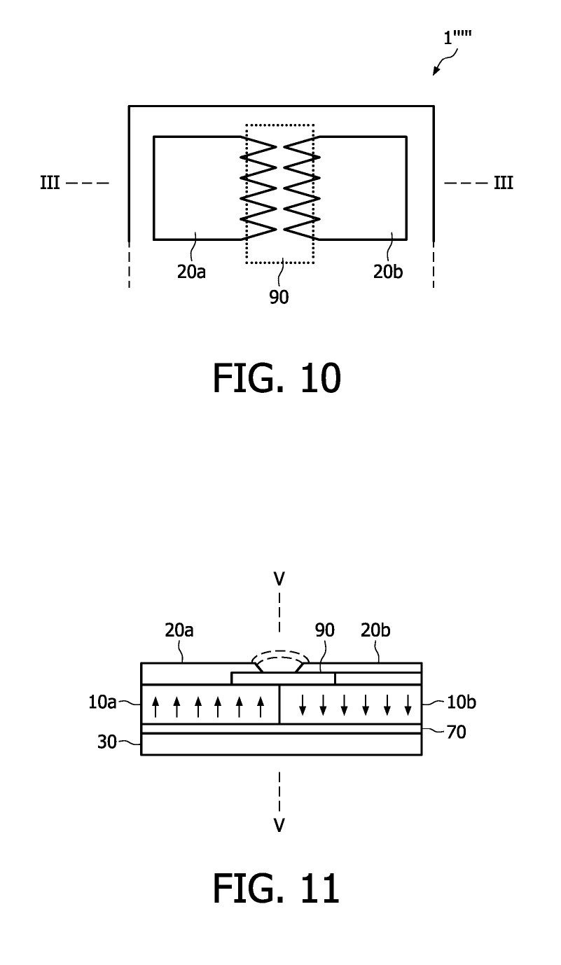

FIG. 10 shows a very schematic partial top view showing a device 1″″′ according to a seventh embodiment of the present invention, viewed along the line V-V in FIG. 11. FIG. 11 shows a very schematic partial cross-sectional view of the device of FIG. 10 along line III-III in FIG. 10.

In the device according to FIGS. 10 and 11, a pyroelectric material layer with two oppositely oriented domains pyroelectric material 10a, 10b is shown. On top of the pyroelectric material, conductive materials 20a, 20b are placed, which form a series of “tips” (as can be seen in FIG. 10).

Between the first conductive materials 20a, 20b, an insulating material 90 is placed. According to a further embodiment (not shown in the figs) the first conductive materials 20a, 20b may be coated with insulating material as well.

The pyroelectric material with the two oppositely oriented domains 10a, 10b is placed on top of a further conductive layer 70 (which does not need to be transparent) and a heating layer 30.

Upon heating, a field is created between the conductive materials 20a, 20b as indicated by the field lines in FIG. 11. The “zig-zag” shape of the conductive materials 20a, 20b leads to a strong concentration of field between the tips, thus allowing a fast and reliable lysis or electroporation of cells.

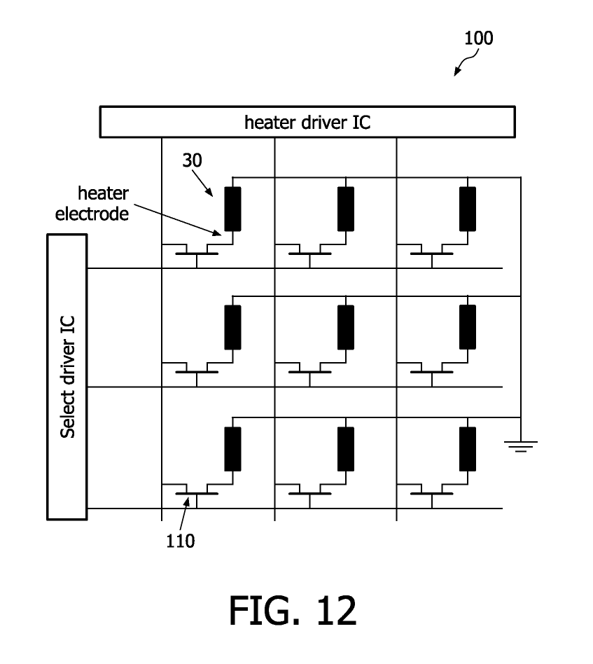

FIG. 12 shows an active matrix heater array for use with a device according to the present invention.

In this embodiment an active matrix 100 is used as a distribution network to route the electrical signals (via transistors 110) required for the heaters 30 from a central driver to the heater elements. Each area of the array may e.g. include a lysing or electroporating structure according to one or more of the previous embodiments. In some embodiments, two heaters (one on the top and one on the bottom) have to be activated simultaneously. This requires either two active matrix plates or vias between the two plates, one being active and the other containing only the heater. For some embodiment only one heater array is needed, which allows a simpler structure.

Sensors may be incorporated in the array to control the performance of the pyroelectric units. The sensors may be thermal sensors or E-field sensors. The signal of the sensors may be going to a processor that monitors the performance of the pyroelectric units or provides a feedback control of the temperature of the units. In another embodiment, the feedback control may be incorporated in an in-pixel electrical circuit.

Preferentially, such an array is realized using large area electronics which are commonly used in the field of LCD and OLED display technologies. In particular, it is advantageous to use the Low Temperature PolySilicon Technology (LTPS).

The particular combinations of elements and features in the above detailed embodiments are exemplary only; the interchanging and substitution of these teachings with other teachings in this and the patents/applications incorporated by reference are also expressly contemplated. As those skilled in the art will recognize, variations, modifications, and other implementations of what is described herein can occur to those of ordinary skill in the art without departing from the spirit and the scope of the invention as claimed. Accordingly, the foregoing description is by way of example only and is not intended as limiting. The invention's scope is defined in the following claims and the equivalents thereto. Furthermore, reference signs used in the description and claims do not limit the scope of the invention as claimed.

1. A cell lysis or electroporation device comprising at least one pyroelectric material.

2. A device according to claim 1, whereby the pyroelectric material is a pyroelectric crystal material.

3. The device according to claim 1, whereby the pyroelectric material is provided at least partially in form of one or more deposited layer(s).

4. The device according to claim 1, whereby the at least one pyroelectric material has an absolute value of the change of charge greater than ≧0.05 mC per ° C. per m².

5. The device according to claim 1, whereby the device furthermore comprises a conductive material in a shape that increases the electrical field.

6. The device according to claim 1, whereby the device furthermore comprises a temperature change means.

7. The device according to claim 1, whereby the at least one pyroelectric material is selected out of the groups:
a) titanates or zirconate titanates, preferably of the structure M^{1I}[ZrₓTi₁₋ₓ]O₃ with 0≦x≦1 and M^{1I} selected out of the group comprising Pb, Be, Mg, Ca, Sr, Ba or mixtures thereof,
b) tantalates, preferably of the structure M^{I}TaO₃ with M¹ selected out of the group comprising Li, Na, K, Rb, Cs or mixtures thereof,
c) niobates, preferably of the structure M^{I}NbO₃ with M¹ selected out of the group comprising Li, Na, K, Rb, Cs or mixtures thereof, or
d) mixtures thereof.

8. A method of lysing or electroporating biological cells comprising the step of applying an electric field generated by a temperature change of at least one pyroelectric material.

9. Use of a pyroelectric material for the lysis or electroporation of biological cells.

10. A system incorporating a device according to claim 1 and being used in one or more of the following applications:
biosensor where cellular components have to be extracted.
biosensor where the porosity of the cell membrane has to be altered.
biosensors used for molecular diagnostics
rapid and sensitive detection of proteins and nucleic acids in complex biological mixtures such as e.g. blood or saliva
high throughput screening devices for chemistry, pharmaceuticals or molecular biology
testing devices e.g. for DNA or proteins e.g. in criminology, for on-site testing (in a hospital), for diagnostics in centralized laboratories or in scientific research
tools for DNA or protein diagnostics for cardiology, infectious disease and oncology, food, and environmental diagnostics
tools for combinatorial chemistry
analysis devices
